(19) United States
(12) Patent Application Publication
(10) Pub. No.: US 2001/0008641 A1
(43) Pub. Date: Jul. 19, 2001

(54) NUTRITIONALLY ACTIVE COMPOSITION FOR BODYBUILDING

(76) Inventor: R. DOUGLAS KROTZER, GALLATIN, TN (US)

Correspondence Address: MARY A. MONTEBELLO, PATTON BOGGS LLP, 2550 M STREET, N.W., WASHINTON, DC, 20037

(21) Appl. No.: 09/199,433

(22) Filed: Nov. 25, 1998

(51) Int. Cl. A61K 35/78

(52) U.S. Cl. 424/725; 424/729; 424/750; 424/734

(57) ABSTRACT

The invention relates to compositions having nutritionally beneficial substituents for bodybuilding. These nutritionally beneficial substituents may be creatine monohydrate, HMB, L-glutamine, taurine, whey peptides, chromium, potassium, phosphorus, and/or magnesium and other substituents that stimulate short and/or long term psychological feedback. The invention also relates to vehicles or devices that accomplish the delivery of the nutritionally beneficial substituent to a recipient.

### FIELD OF THE INVENTION

The invention relates to compositions having nutritionally beneficial substituents to enhance or facilitate bodybuilding and substituents that stimulate short and/or long term psychological feedback. The invention also relates to vehicles or devices that accomplish the delivery of the nutritionally beneficial substituents to a recipient. The invention particularly concerns compositions for oral or translingual delivery using beverages, lozenges and the like as vehicles to facilitate delivery. The invention additionally concerns the use of transdermal delivery devices (e.g., patches) to accomplish the delivery of such substituent(s). The invention particularly concerns a formulated composition, preferably a beverage for oral consumption, having a nutritionally beneficial substituent and a substituent that stimulates psychological feedback and which accomplishes the delivery of the nutritionally beneficial substituent to enhance or facilitate bodybuilding.

### BACKGROUND OF THE INVENTION

For many individuals, adhering to a daily routine of nutritional supplementation is difficult; it may require a change of habit, or practices, by the consumer. More significantly, nutritional agents may not cause an immediately discernible effect, and, lacking this or other positive feedback, the consumer may discontinue use prematurely. The present invention is directed to this problem.

There are many examples of beverages providing nutritional supplements. U.S. Pat. No. 5,626,849 (Hastings et al.) concerns a composition for facilitating weight loss. The composition contains chromium, L-carnitine, γ-linolenic acid, (-) hydroxycitric acid, choline, inositol, antioxidants and herbs. U.S. Pat. No. 5,567,424 (Hastings) describes a beverage composition containing herbs, fiber, antioxidants and enzymes. U.S. Patent 5,536,506 (Majeed et al.) describes piperine-containing compositions. U.S. Pat. No. 5,290,605 (Shapira) concerns a nutritional soft drink whose ingredients are said to provide protection from UV damage. The composition contains a carotenoid mix and optionally, fruit/vegetable juices and/or herbal preparations. U.S. Pat. No. 5,240,732 (Ueda) concerns plant extract-containing beverages supplemented with a sugar-alcohol.

Nutritional supplements having psychological feedback properties have also been described. U.S. Pat. No. 5,681,569 (Kuznicki et al.) and 4,946,701 (Tsai et al.) both concern beverage compositions that contain green tea extracts. U.S. Pat. No. 5,674,522 (Shah et al.) concerns a powdered concentrate containing one or more pharmacologically active agents for use in hot beverages. The composition may contain caffeine, as well as vitamins and minerals. U.S. Pat. No. 5,571,441 (Andon et al) is directed to nutritional supplements compositions that provide psychological feedback.

The disclosed compositions contain vitamins and/or minerals as well as xanthine alkaloids (such as caffeine, etc.) that provide a physiological signal. U.S. Pat. No. 5,114,723 (Stray-Gundersen) concerns hypotonic beverages for supplying physiologically essential electrolytes, nutrient minerals, carbohydrates and other ingredients to a consumer. The patent discloses that caffeine may be added to the beverage formulation. U.S. Pat. No. 4,992,282 (Mehansho et al.) concerns vitamin and mineral-fortified beverages, which may be supplemented with caffeine. U.S. Pat. No. 4,612,205 (Kupper et al.) concerns fruit-flavored carbonated beverages that may be supplemented with caffeine. U.S. Pat. No. 4,061,797 (Hannan, Jr. et al.) concerns non-carbonated, caffeinated fruit beverages.

U.S. Pat. No. 4,737,375 (Nakel et al.) describes calcium-supplemented beverages. U.S. Pat. No. 4,769,244 (Lavie) describes non-hygroscopic water-soluble pulverulent compositions that may be employed to make beverages.

Compositions for accomplishing transdermal delivery of pharmaceuticals have also been described (see, for example, U.S. Pat. Nos. 5,718,914 (Foldvari), 5,698,217 (Wilking), 5,721,257 (Baker), 5,683,713 (Blank etal), and 4,788,189 (Glazer)).

The present invention provides an improved composition for accomplishing the delivery of nutritionally beneficial substituents to enhance or facilitate bodybuilding. In a preferred embodiment, the present invention additionally provides an improved formulated beverage that provides at least one, and most preferably multiple, short and long term psychological feedback(s) to the consumer.

### SUMMARY OF THE INVENTION

Conventional or traditional beverages are formulated to provide external sensory appeal (such as taste, sight, or smell). In contrast to such compositions, the present invention provides beverages and other compositions for bodybuilding that are formulated to provide internal appeal directly to the brain (i.e., brain rewards). The present invention thus relates to a formulated, defined composition having a nutritionally beneficial substituent to enhance or facilitate bodybuilding and a long and/or short term substituent that stimulates psychological feedback and to vehicles or devices that accomplish the delivery of the nutritionally beneficial substituent. Such preferred compositions will typically possess a nutritionally beneficial substituent and a substituent that stimulates both a long term and a short term psychological feedback and which accomplishes the delivery of the nutritionally beneficial substituent. Preferred vehicles include oral and/or translingual delivery vehicles, such as beverages, elixirs, lozenges, chewable tablets, and the like, and transdermal delivery devices such as patches, bandages, etc. As used herein, the term “bodybuilding” refers to the strengthening and toning of body muscles, the decreasing of an individual's ratio of body fat to muscle, an improvement in the appearance of muscle strength and tone, and/or an improvement in an individual's overall feeling of well-being and appearance associated with strengthening and toning of the muscles of the body.

In detail, the invention provides a composition (preferably a beverage, and more preferably a substantially alcohol-free beverage, for human consumption) comprising:

(A) a nutritionally beneficial substituent selected from the group consisting of:

creatine monohydrate, HMB, L-glutamine, taurine, whey peptides, chromium, potassium, phosphorus, and magnesium,

the substituent present in an amount sufficient to enhance or facilitate bodybuilding; and

(B) at least one additional substituent that provides traditional psychological feedback selected from the group consisting of

caffeine or a caffeine equivalent, tryptophan, ephedra, cola, green tea extract, carbonic acid, phosphoric acid, citric acid, hops, cocoa, chocolate, an anandamide, quinine, malic acid, a sweetener, a fruit juice or fruit juice extract, milk, a vegetable juice or vegetable juice extract, kudzu and 5-hydroxy-tryptophan,

present in an amount sufficient to provide a sensory psychological feedback.

The invention further provides such compositions that additionally contain at least one additional substituent (C) that provides long term psychological feedback substituent and/or at least one additional substituent (D) that provides short term psychological feedback; wherein

the substituent (C) that provides the long term psychological feedback is selected from the group consisting of:

an anandamide, 5-hydroxytryptophan, 5-fluoro-A-methyltryptamine, 5-fluorotryptophan, 6-fluorotryptophan, tryptophan, allocryptine, caffeine, theophylline, theobromine, California poppy, calcium, chromium picolinate, chromium polynicotinate, chicalote extract, cocoa, chocolate, Damiana (Turnera diffusa), DL-phenylalanine, ephedra, ephedrine, epinephrine, GABA, ginger, ginseng, L-glutamine, green tea, guarana, kava kava, lactuca virosa, L-tyrosine, lobelia, magnesium, maraba, protopine, pseudophedrine, pseudo-epinephrine, pyridoxal-5-phosphate, red rice yeast, serotonin, sucrose, fructose, glucose, high fructose corn syrup, and St. Johnswort, and

is present in an amount sufficient to provide a long term feeling of well-being or calmness; and the substituent (D) that provides the short term psychological feedback is selected from the group consisting of:

an anandamide, an alcohol enhancer, angelica root, balm, bitter orange (Auranti pericarpium), bogbean, boldo, calamus, California poppy, capsaicum, caraway, cayenne, chamomile, cinchona bark, quinine, chocolate, cinnamon, clove, cocoa, condurango, dandelion, elecampane, GABA, gentian, ginger, ginseng, holy thistle, hops, horehound, dried lemon peel (Citri pericardium), mugwort, unripe orange, peppermint, quassia, red sage, rosemary, star anise, thyme, tumeric, wormwood, yarrow, and zinc, and is present in an amount sufficient to provide a short term sensation of warmth, tingling, excitement, tranquility and well-being, or a distinctive, intense, bitter or unusual taste.

The invention additionally provides a method for enhancing or facilitating bodybuilding in a human, which comprises administering or providing to the human a composition comprising:

at least one nutritionally beneficial substituent (A) selected from the group consisting

of:

creatine monohydrate; HMB; L-glutamine; taurine; whey peptides;

chromium; potassium; phosphorus; and magnesium;

the substituent being present in an amount sufficient to enhance or facilitate bodybuilding of the human recipient; and at least one additional substituent (C) that provides long term psychological feedback substituent or at least one additional substituent (D) that provides short term psychological feedback; wherein the substituent (C) that provides the long term psychological feedback is selected from the group consisting of:

an anandamide; 5-hydroxytryptophan; 5-fluoro-A-methyltryptamine; 5-fluorotryptophan; 6-fluorotryptophan; tryptophan; allocryptine; caffeine; theophylline; theobromine; California poppy; calcium; chromium picolinate; chromium polynicotinate; chicalote extract; cocoa; chocolate; Damiana (Turnera diffuse); DL-phenylalanine; ephedra; ephedrine; epinephrine; GABA; ginger; ginseng; L-glutamine; green tea; guarana; kava kava; lactuca virosa; L-tyrosine; lobelia; magnesium; maraba; protopine; pseudophedrine; pseudoepinephrine; pyridoxal-5-phosphate; red rice yeast; serotonin; sucrose; fructose; glucose; high fructose corn syrup; and St. Johnswort; and

is present in an amount sufficient to provide a long term feeling of well-being or calmness; and the substituent (D) that provides the short term psychological feedback is selected from the group consisting of:

an anandamide; an alcohol enhancer; angelica root; balm; bitter orange (Auranti pericarpium); bogbean; boldo; calamus; California poppy; capsaicum; caraway; cayenne; chamomile; cinchona bark; quinine; chocolate; cinnamon; clove; cocoa; condurango; dandelion; elecampane; GABA; gentian; ginger; ginseng; holy thistle; hops; horehound; dried lemon peel (Citri pericardium); mugwort; unripe orange; peppermint; quassia; red sage; rosemary; star anise; thyme; tumeric; wormwood; yarrow; and zinc; and

is present in an amount sufficient to provide a short term sensation of warmth, tingling, excitement, tranquility and well-being, or a distinctive, intense, bitter or unusual taste.

### DETAILED DESCRIPTION OF THE INVENTION

The invention relates to formulated, defined compositions having a nutritionally beneficial substituent for bodybuilding and a long and/or short term substituent that stimulates psychological feedback, optionally in combination with a substituent that provides a traditional psychological feedback. The invention further relates to vehicles or devices that accomplish the delivery of the composition to a recipient. In a more preferred embodiment, the invention concerns such formulated, defined compositions that contain both a short term and a long term substituent that stimulates psychological feedback.

### THE DELIVERY VEHICLES OR DEVICES OF THE INVENTION

The compositions of the present invention are formulated or included in vehicles or devices that accomplish their delivery to recipients, and in particular, to recipient humans. Vehicles suitable for oral or translingual delivery are particularly preferred. A translingual delivery vehicle is one a that accomplishes the delivery of the composition via adsorbtion into the tongue, gums or soft tissues of the oral cavity. A vehicle suitable for oral delivery is one that accomplishes the delivery of the composition after ingestion. Oral and translingual/transdermal vehicles include beverages, lozenges, elixirs, syrups, powders, candies, chewable tablets, etc.

In an alternate embodiment, the delivery of the compositions of the present invention is accomplished using a transdermal delivery device. Such devices include bandages, patches, implants, etc. that accomplish the delivery of substituents via adsorption through the skin.

The compositions of the present invention are said to be “formulated, defined compositions” by which it is meant that at least two, and preferably all, of their chemical substituents are substantially defined chemically. Thus, although the compositions of the present invention may contain undefined substituents, such as those found in naturally obtainable plant extracts, etc., in all instances they contain at least two chemically ascertainable substituents that were deliberately added to the composition in predetermined amounts to achieve preselected concentrations. Where appropriate, the amounts of such substituents in the compositions of the invention will preferably be determined with consideration of the beneficial concentration (potency) of the substituent.

Beverages for oral administration are the preferred delivery vehicle. As used herein, the term “beverage”is intended to refer to a liquid composition that is in “single” strength form and is ready to drink. Examples of beverage compositions are well known (see, U.S. Pat. Nos. 4,737,375 (Nakel et al), 4,992,282 (Mehansho et al.), 5,683,678 (Heckert et al.), 5,571,441 (Andon etal.), each herein incorporated by reference).

The invention contemplates the formulation and use of such beverages, as well as the formulation and use of beverage “concentrates.” Such concentrates include solid materials (capsules, tablets, powders, etc.) or solutions, suspensions, or other liquids that require dilution before attaining their desired final state. Such liquid compositions may be formulated from the dissolution or suspension of solid substituents, or from the mixing of liquid substituents, or both. The liquid compositions of the present invention are intended to be orally provided, as distinguished from intravenous or other means.

The beverages of the present invention may be imbibed cooled (as by a refrigerant), or at ambient or elevated temperature (i.e., from about 25° C. to about 50° C.) or at more elevated temperatures (i.e., as a “hot” drink). In a preferred embodiment, the beverages of the present invention are imbibed at temperatures below 180° C. In a more preferred embodiment, the beverages of the present invention are imbibed at temperatures below 25°C.

The beverages of the present invention are preferably alcohol-free. They thus contain less than about 10% ethanol, more preferably less than about 5% ethanol, and most preferably are alcohol-free (i.e. only a trace amount of ethanol or none at all). The term “alcohol-free,” as used herein, is intended to be limited to consideration of the ethanol (ethyl alcohol) concentration of the beverage, and not to refer generally to concentrations of other non-ethanol alcohols that may be present. In an alternative embodiment, the beverages of the present invention may be alcohol-containing beverages (i.e., a beverage having between about 10% and about 40% alcohol).

The beverages of the present invention may be either carbonated or non-carbonated. The term “carbonated” means that the beverage contains both dissolved and dispersed carbon dioxide. Methods of forming carbonated beverages are well known in the art (see, for example, U.S. Pat. No. 4,946,701 (Tsai etal.). Usually, when carbonated, the beverages of the present invention will contain from 1.0 to about 4.5 volumes of carbon dioxide. The preferred carbon dioxide beverages contain from about 2 to about 3.5 volumes of carbon dioxide.

The compositions of the present invention contain at least one substituent selected from a first class of substituents that comprise nutritionally beneficial compounds for bodybuilding. Any of a wide array of such substituents may be included in the compositions of the invention. The compositions of the present invention contain at least one, and more preferably 2 or more substituents of this first class. Preferred substituents of this first class, their associated function, and their preferred concentrations in the compositions of the present invention are described in Table A. To be nutritionally beneficial, as used herein, a substituent is to be provided at a concentration sufficient to impart a nutritional benefit to the recipient. The preferred daily intake amount of nutritionally beneficial amounts of substituent is provided in Table A. The daily intake amount may be obtained by a single serving, etc., or by multiple servings, etc. In a preferred embodiment, the daily intake amount will be delivered in three servings, each comprising one third of the total preferred intake amount. For a beverage, examples of typical serving sizes are any size between 1 and 16 fluid ounces (e.g., 16 ounces, 12 ounces, 8 ounces, 6 ounces, or 1 ounce) or metric equivalents. In Table A, the amount/day shown is the most preferred amount per day, unless a maximum, more preferred and/or most preferred amount/day is indicated. Where no amount/day is provided, the substituent may be provided in any customary or traditional amount/day.

TABLE A
Nutritionally Beneficial Substituents

| | | Amount/Day | | | |
|---|---|---|---|---|---|
| | | | More | Most | Associated |
| Substituent | Units | Maximum | Preferred | Preferred | Function(s) |
| Creatine | Mg | 910- | 12,890- | 29,990 | Daily recommended |
| Monohydrate | | 150,050 | 60,000 | | dose during loading |
| | | | | | phase |
| | | 134- | 2,220- | 7,510 | Daily recommended |
| | | 125,010 | 44,900 | | dosage for |
| | | | | | maintenance |
| HMB (beta- | Mg | 67-27,890 | 1,010- | 4,050 | Up-regulates ability to |
| hydroxy | | | 8,980 | | build muscle and |
| beta- | | | | | burn fat in response |
| methylbutyrate) | | | | | to intense exercise; |
| | | | | | decreases stress- |
| | | | | | induced muscle |
| | | | | | protein breakdown |
| L-Glutamine | Mg | 37-19,010 | 540-5,990 | 1,910 | |
| Taurine | Mg | 18-48,525 | 230- | 752 | |
| | | | 16,440 | | |
| Whey Peptides | Mg | 55-55,650 | 845- | 6,440 | Yields the most usable |
| | | | 18,990 | | grams for amino |
| | | | | | acids than other |
| | | | | | protein supplements |
| Chromium | Mcg | 4-19,998 | 52-3,700 | 1,100 | |
| Potassium | Mg | 35-27,640 | 490-9,770 | 2,300 | |
| Phosphorus | Mg | 16-25,450 | 230-9,830 | 2,690 | |
| Magnesium | Mg | 10-5,435 | 110-1,960 | 498 | |

In a preferred embodiment of the invention, the compositions of the present invention will contain one or more of such nutritionally beneficial substituents.

More Preferred Nutritionally Beneficial Substituents: In a more preferred embodiment, the compositions of the present invention will contain one or more preferred nutritionally beneficial substituents, such more preferred substituents being selected from the group consisting of creatine monohydrate, HMB, L-glutamine, taurine.

Highly Preferred Nutritionally Beneficial Substituents: In a still more preferred embodiment, the compositions of the present invention will contain one or more highly preferred nutritionally beneficial substituents, such highly preferred substituents being selected from the group consisting of creatine monohydrate, and HMB.

In a further optional embodiment, the composition of the invention may contain one or more additional nutritionally beneficial substituents.

The compositions of the invention may additionally contain at least one substituent selected from a second class of substituents that provide a traditional psychological feedback. Any of a wide array of such substituents may be included in the composition. Such substituents will impart a sensory psychological effect to the recipient, such as a pleasant taste, aroma, visual appeal, etc. The compositions of the invention may contain more than one such traditional psychological feedback substituent. Table B lists preferred traditional psychological feedback substituents that may be employed in the compositions of the present invention to impart such psychological effect.

TABLE B
Preferred Traditional Psychological Feedback Substituents

| | Concentration |
|---|---|
| Substituent | (amount/day) |
| Caffeine and caffeine equivalents (e.g., theophylline, theobromine, | To Limit Of Non- |
| related methylxanthines) | Prescription Medication |
| Green Tea Extract | |
| Carbonic Acid | |
| Phosphoric Acid | |
| Citric Acid | |
| Hops | |
| Cola | |
| Cocoa | |
| Chocolate | |
| Anandamide | |
| Quinine | |
| Malic Acid | |
| Sweetener (such as caloric sweeteners (e.g., fructose, high | |
| fructose corn syrup, sucrose, maltose, glucose, lactose, sorbitol, | |
| galactose, etc.) or substantially non-caloric sweeteners (e.g., | |
| aspartame (and its derivatives), saccharin, L-sugars, cyclamates, | |
| etc.) | |
| Fruit juice or juice extract such as non-citrus fruit juices or juice | |
| extracts (e.g., those obtainable from apple, pineapple, grape, pear, | |
| banana, plum, cherry, peach, etc.); berry fruit juices or juice | |
| extracts (such as those obtainable from strawberry, blueberry, | |
| cranberry, blackberry, etc.); or citrus fruit juices or juice extracts | |
| (such as those obtainable from orange, grapefruit, lemon, lime, | |
| etc.) | |
| Milk (including mammalian milk products such as skim milk and | |
| cream, and vegetable milks, such as soy milk) | |
| Vegetable juices or juice extracts (such as those obtainable from | |
| carrot, tomato, beets, celery, etc.) | |
| kudzu | |
| 5-hydroxy-tryptophan | |
| Spices and plant extract flavorings (such as cola, ginger, nutmeg, | |
| pepper, vanilla, chocolate, rum, mint, sugar maple, herbs, etc.). | |

Synthetically compounded flavorants or naturally isolated flavorants may be employed.

As indicated, the orally or translingually delivered compositions of the present invention may be formulated to contain green tea extract. Green tea helps to control the negative effects of caffeine. See, for example, French patent No. 2,586,532 issued to Balansard et al.

More Preferred Traditional Psycholociical Feedback Substituents: In a more preferred optional embodiment, the compositions of the present invention will contain one or more preferred traditional psychological feedback substituents, such more preferred substituents being selected from the group consisting of: Caffeine and caffeine equivalents (e.g., theophylline, theobromine, related methylxanthines); cola; carbonic acid; phosphoric acid; citric acid; hops; cocoa; chocolate; anandamide; quinine; malic acid; non-citric fruit juice or juice extract (such as wherein the fruit is grape, apple, cranberry, cherry, peach, etc.); milk; vegetable juice or juice extract (such as wherein the vegetable is carrot, celery, etc.); kudzu; and 5-hydroxy-tryptophan.

Highly Preferred Traditional Psychological Feedback Substituents: In a still more preferred embodiment, the compositions of the present invention will contain one or more highly preferred traditional psychological feedback substituents, such highly preferred substituents being selected from the group consisting of: Caffeine and caffeine equivalents (e.g., theophylline, theobromine, related methylxanthines) at a concentration/day of greater than about 65 mg; carbonic acid; phosphoric acid; hops; non-citric fruit juice or fruit juice extract (such as wherein the fruit is grape, apple, cranberry, cherry, peach, etc.); milk; vegetable juice or juice extract (such as wherein the vegetable is carrot, celery, etc.); kudzu; and 5-hydroxy-tryptophan.

The compositions of the invention may additionally contain at least one substituent selected from a third class of substituents that provide long term psychological feedback. Any of a wide array of such substituents may be included in the composition. Such substituents will impart a long term psychological effect to the recipient, such as a feeling of warmth or tingling, of excitement, of tranquillity and well-being, etc. In a preferred embodiment, the compositions of the invention will contain more than one such long term psychologically active substituent. Table C lists particularly preferred long term duration psychological feedback substituents that may be employed in the compositions of the present invention to impart a long term psychological effect.

TABLE C
Preferred Long Term Psychological Feedback Substituents

Concentration (amount/day)

| | | | More | Most | Associated | | |
|---|---|---|---|---|---|---|---|
| Substituent | Units | Maximum | Preferred | Preferred | Function(s) | | |
| Anandamide | mg | 31-296,000 | 1,013- | 7,083 | Psychological Feedback | | |
| | | | 144,600 | | Warm feeling | | |
| | | | | | Euphoria | | |
| 5-hydroxytryptophan | mg | 5-18,000 | 60-3,468 | 357 | Decrease Alcohol Desire | | |
| or equivalent | | | | | Mood Stabilization | | |
| compounds | | | | | Increases Brain | | |
| (e.g., 5-fluoro-A- | | | | | Serotonin | | |
| methyltryptamine, 5- | | | | | Reduces Withdrawal | | |
| fluorotryptophan, 6- | | | | | Stresses | | |
| fluorotryptophan, | | | | | Restores Deficit | | |
| tryptophan, etc.) | | | | | Associated With | | |
| | | | | | Alcohol | | |
| Allocryptine | mg | 0.1-50,400 | 5-1,800 | 170 | Reduce Reward Effect | | |
| | | | | | of Alcohol | | |
| | | | | | Calming | | |
| | | | | | Relieves Withdrawal | | |
| | | | | | Symptoms from | | |
| | | | | | both Depressants | | |
| | | | | | and Stimulants | | |
| Caffeine (including | mg | 2-1000 | 36-723 | 322 | Discourage Alcohol Use | | |
| (theophylline, | | | | | Increase Alcohol | | |
| theobromine and | | | | | Metabolism | | |
| related | | | | | Inhibit Adenosine | | |
| methylxanthines) | | | | | Uptake | | |
| (amounts are total | | | | | Stimulant | | |
| effective available | | | | | | | |
| amounts of caffeine | | | | | | | |
| in composition) | | | | | | | |
| California Poppy | mg | 0.1-50,400 | 5-1,800 | 170 | Reduce Reward Effect | | |
| | | | | | of Alcohol | | |
| | | | | | Calming | | |
| | | | | | Relieves Withdrawal | | |
| | | | | | Symptoms from both | | |
| | | | | | Depressants and | | |
| | | | | | Stimulants | | |
| Calcium | mg | 10-4,000 | 567-2,980 | 1,222 | Alleviate Depression | | |
| Chromium | μg | 2-800 | 5-400 | 194 | Increases Tryptophan | | |
| Cr-Picolinate | | | | | Blood/Brain Barrier | | |
| Cr-Polynicotinate | | | | | Transport | | |
| (Cr amounts | | | | | | | |
| increased with | | | | | | | |
| amount of Zn | | | | | | | |
| present, decreased | | | | | | | |
| for amounts of | | | | | | | |
| amino acids) | | | | | | | |
| Chicalote Extract | μg | 2-800 | 5-400 | 194 | Reduce Reward Effect | | |
| (without toxic | | | | | of Alcohol | | |
| substituents) | | | | | Calming | | |
| | | | | | Relieves Withdrawal | | |
| | | | | | Symptoms from both | | |
| | | | | | Depressants and | | |
| | | | | | Stimulants | | |
| Cocoa | mg | 31-300,000 | 1,013- | 7,083 | Activates Cannabinoid | | |
| | | | 145,000 | | Receptors | | |
| | | | | | Warm Feeling | | |
| | | | | | Euphoria | | |
| Chocolate | mg | 31-300,000 | 1,013- | 7,083 | Activates Cannabinoid | | |
| | | | 145,000 | | Receptors | | |
| | | | | | Warm Feeling | | |
| | | | | | Euphoria | | |
| Damiana (Turnera | Tbsp | 0.5-15 | 1-8 | 6 | Mild Aphrodisiac | | |
| diffusa) | | | | | Mild Euphoria (1-1.5 | | |
| | | | | | hours) | | |
| | | | | | Decrease Alcohol Desire | | |
| DL-phenylalanine | g | 0.01-12 g | 0.4-9 g | 2.3 g | Psychological feedback | | |
| | | | | | Mood Stabilization | | |
| | | | | | Decrease Sugar Desire | | |
| | | | | | Decrease Alcohol Desire | | |
| | | | | | Decrease Chronic Pain | | |
| Ephedra (especially | mg | 6-8,000 | 61-2,990 | 376 | Stimulant | | |
| nevadenis) | | | | | Decrease Alcohol Desire | | |
| Ephedrine | mg | 0.9-390 | 6-149 | 23.6 | Stimulant | | |
| | | | | | Decrease Alcohol Desire | | |
| Epinephrine | | | | Legal | Stimulant | | |
| | | | | Amounts | | | |
| GABA | g | 0.75-39 | 3.6-14.3 | 7.43 | Promote Calmness | | |
| | | | | | Tingling Sensation on | | |
| | | | | | Skin | | |
| Ginger | g | 0.026-10 | 0.55-5 | 2.17 | Antioxidant | | |
| | | | | | Blood Sugar Stabilizer | | |
| | | | | | Controls Nausea | | |
| | | | | | Stipulate Appetite | | |
| | | | | | Aromatic Smell | | |
| | | | | | Flush, Rosy complexion | | |
| Ginseng | g | 0.05-20 | 0.9-4.9 | 1.6 | Stimulant | | |
| | | | | | Enhances Mental | | |
| | | | | | Concentration | | |
| | | | | | Energizes | | |
| L-glutamine | mg | 111-8,891 | 556-4,808 | 2,746 | Blood Sugar Stabilizer | | |
| | | | | | Promote Calmness | | |
| | | | | | Reduces Stress of | | |
| | | | | | Alcohol Withdrawal | | |
| | | | | | Suppress Alcohol Desire | | |
| | | | | | Suppress Appetite | | |
| Green Tea | g | 0.05-25 | 0.9-19 | 8.80 | Promote Calmness and | | |
| | | | | | Relaxation | | |
| | | | | | Decreases Alcohol in | | |
| | | | | | Blood | | |
| | | | | | Delays Caffeine | | |
| | | | | | Stimulation | | |
| | | | | | Blood Sugar Stabilizer | | |
| Guarana | g | 0.2-3.4 | 1.2-2.7 | 2.06 | Increases Alcohol | | |
| | | | | | Metabolism | | |
| | | | | | Reduces Blood Sugar | | |
| | | | | | Suppresses Alcohol | | |
| | | | | | Desire | | |
| Kava Kava | | | | | Relaxes Muscles | | |
| | | | | | Calming | | |
| Lactuca Virosa | μg | 0.01-10,000 | 1.4-4,569 | 234 | Calming | | |
| | | | | | Contains Trace Amounts | | |
| | | | | | of Morphine | | |
| L-tyrosine | g | 0.001-8 | 0.05-3.9 | 2.6 | Psychological feedback | | |
| | | | | | Neurotransmitter (L- | | |
| | | | | | dopa) precursor | | |
| Lobelia | mg | 58-6,978 | 356-1,367 | 745 | Psychological feedback | | |
| | | | | | Decreases Desire for | | |
| | | | | | Alcohol | | |
| Magnesium | mg | 1-1,990 | 108-879 | 546 | Decrease Alcohol | | |
| | | | | | Consumption | | |
| | | | | | Restore Deficit | | |
| | | | | | Associated with | | |
| | | | | | Alcohol Consumption | | |
| | | | | | Eases Withdrawal | | |
| | | | | | Symptoms of | | |
| | | | | | Hyperexcitability and | | |
| | | | | | Hallucination | | |
| | | | | | Mood Stabilization | | |
| Maraba (Red Wheat | | | | | | | |
| Fermented) | | | | | | | |
| Protopine | mg | 0.1-50,400 | 5-1,800 | 170 | Reduce Reward of | | |
| | | | | | Alcohol | | |
| | | | | | Calming | | |
| | | | | | Relieves Withdrawal | | |
| | | | | | Symptoms for both | | |
| | | | | | Depressants and | | |
| | | | | | Stimulants | | |
| Pseudophedrine | mg | 0.01-79 | 0.09-24 | 9.8 or | Stimulant | | |
| | | | | | | maximum | Decrease Alcohol Desire |
| | | | | legal | | | |
| | | | | amount | | | |
| Pseudoepinephrine | mg | maximum | Stimulant | | | | |
| | | | | legal | Decrease Alcohol Desire | | |
| | | | | amount | | | |
| Pyridoxal-5- | mg | 1-989 | 31-199 | 124 | Decreases Alcohol | | |
| | | | | | Desire | | |
| phosphate | | | | | Alleviate Depression | | |
| | | | | | Enable | | |
| | | | | | Neurotransmitters | | |
| | | | | | Mood Stabilization | | |

| | | |
|---|---|---|
| Serotonin/ | (generated in the Brain) | Psychological feedback |
| Enkaphaline | | |

| | | | | | |
|---|---|---|---|---|---|
| Sucrose, Fructose, | | 1.5-30% | 7.5-17.4% | 10.11% | Discourage use of |
| Glucose, High | | | | | alcohol |
| Fructose Corn Syrup | | | | | Decreases Duration of |
| | | | | | Alcohol Action |
| | | | | | Inhibits Tryptophan |
| | | | | | Destruction |
| St. Johnswort | g | 0.4-11 | 1.2-6.9 | 3.2 | Suppress Appetite |
| | | | | | Feels Good |
| | | | | | Monomine Oxidase |
| | | | | | Inhibitor |
| | | | | | Eases Withdrawal |
| | | | | | Suppress alcohol Desire |
| Red Rice Yeast | | | | | Mood Stabilizing |
| | | | | | Calming |
| Vitamin B6 | mg | 0.01-200 | 0.8-49.8 | 1.45 | Mood Stabilization |
| | | | | | Immune System |
| | | | | | Support |

More Preferred Long Term Psychological Feedback Substituents: In a preferred embodiment, the compositions of the present invention will contain one or more such long term psychological feedback substituents, such more preferred substituents being selected from the group consisting of: 5-hydroxytryptophan or equivalent compounds (e.g., 5-fluoro-A-methyltryptamine, 5-fluorotryptophan, 6-fluorotryptophan, tryptophan, etc.); Allocryptine; California Poppy; Chromium (Cr-Picolinate; Cr-Polynicotinate; (Cr amounts increased with amount of Zn present, decreased for amounts of amino acids)); Cocoa; Damiana (Turnera diffuse); D-phenylalanine; Ephedra (especially nevadenis); Ephedrine; Epinephrine; GABA; L-10 glutamine; Green Tea; Kava Kava; Lactuca Virosa; Lobelia; Magnesium; Maraba; Protopine; Pseudophedrine; Pseudoepinephrine; Pyridoxal-5-phosphate; Red rice yeast; Serotonin; St.

Johnswort; Vitamin B6.

Highly Preferred Long Term Psychological Feedback Substituents: In a still more preferred embodiment, the compositions of the present invention will contain one or more such long term psychological feedback substituents, such highly preferred substituents being selected from the group consisting of: 5-hydroxytryptophan or equivalent compounds (e.g., 5-fluoro-A-methyltryptamine, 5-fluorotryptophan, 6-fluorotryptophan, tryptophan, etc.);

Allocryptine; California Poppy; Chicalote Extract (without toxic substituents); Cocoa; Damiana (Turnera diffusa); Ephedra nevadenis; Ephedrine; Green Tea; Lactuca Virosa; Lobelia; Maraba; Protopine; Pseudophedrine; Pseudoepinephrine and Red rice yeast.

The compositions of the invention may additionally contain at least one substituent selected from a fourth class of substituents that provide short term psychological feedback. Any of a wide array of such substituents may be included in the composition. Such substituents will impart a short term psychological effect to the recipient, such as a feeling of warmth or tingling, of excitement, of tranquillity and well-being, etc. In a preferred embodiment, the compositions of the invention will contain more than one such short term psychologically active substituent.

In a particularly preferred embodiment, the compositions of the invention will contain psychologically active substituents that mediate their respective effects in both a short duration (i.e., effect completed within 15 minutes of nutritionally beneficial.) and a long term duration (i.e., effect completed after 15 minutes of nutritionally beneficial). Table D lists preferred short term duration psychological feedback substituents that may be employed in the compositions of the present invention to impart a short term psychological effect.

TABLE D
Preferred Short Term Psychological Feedback Substituents

| | | Concentration (amount/day) | | | |
|---|---|---|---|---|---|
| | | | More | Most | Associated |
| Substituent | Units | Maximum | Preferred | Preferred | Function(s) |
| Anandamide | mg | 31-296,000 | 1,013- | 7,083 | Warm feeling |
| | | | 145,000 | | Euphoria |
| Alcohol | | Between just noticeable and | | | Imitate Burning and |
| Enhancers, | | disagreeable | | | Biting Sensations of |
| Standard | | | | | Alcohol |
| Flavorants | | | | | |
| Angelica root | | | | | Bitter Aromatic |
| | | | | | Initially Intensely Spicy |
| | | | | | Turns Lastingly Pungent |
| | | | | | Stimulates Appetite |
| Balm | | | | | Calming |
| | | | | | Pleasantly Spicy Taste |
| Bitter orange | | | | | Stimulate Appetite |
| (Auranti | | | | | Pleasantly Spicy Taste |
| pericarpium) | | | | | |
| Bogbean | | | | | Stimulate Appetite |
| | | | | | Stimulate Gastric Acid |
| Boldo | | | | | Weak Hypnotic |
| | | | | | Pungently Spicy, Bitter |
| | | | | | Taste |
| Calamus | | | | | Somewhat Spicy |
| | | | | | Calming |
| California Poppy | mg | 0.1-50,400 | 5-1,800 | 170 | Euphoriant |
| | | | | | Reduce Reward Effect |
| | | | | | of Alcohol |
| Capsaicum | | 0.001-1.0% | 0.01-0.6% | 0.025% | Warming Sensation in |
| | | | | | Throat and Mouth |
| Caraway | | | | | Stimulate Appetite |
| | | | | | Relieves Flatulence |
| | | | | | Caused by Carbonation |
| | | | | | Spicy Taste |
| Cayenne | | 0.001-1% | 0.01-0.6% | 0.025% | Warming Sensation in |
| | | | | | Throat and Mouth |
| Chamomile | | | | | Stimulate Appetite |
| | | | | | Aromatic Taste |
| Cinchona bark | | | | | Astringent Taste |
| (quinine) | | | | | Stimulate Appetite |
| Chocolate | mg | 31-296,000 | 1,013- | 7,087 | Psychological feedback |
| | | | 157,000 | | Warm feeling |
| | | | | | Euphoria |
| Cinnamon | | | | | Stimulate Appetite |
| | | | | | Pungently Sweet |
| | | | | | Sweep, Sharp Taste |
| | | | | | Relieves Flatulence |
| | | | | | Associated with |
| | | | | | Carbonation |
| Clove | | | | | Stimulate Appetite |
| | | | | | Pungently Sweet |
| | | | | | Relieves Flatulence |
| | | | | | Associated with |
| | | | | | Carbonation |
| Cocoa | | | | | Psychological Feedback |
| | | | | | Warm Feeling |
| | | | | | Euphoria |
| Condurango | | | | | Stimulate Appetite |
| | | | | | Bitter Taste |
| Dandelion | | | | | Slight Bitter Taste |
| | | | | | Stimulate Appetite |
| Elecampane | | | | | Stimulate Appetite |
| | | | | | Relieves Flatulence |
| | | | | | Associated with |
| | | | | | Carbonation |
| GABA | g | 0.75-39 | 3.6-14.3 | 7.43 | Tingling Sensation on |
| | | | | | Skin After Taking |
| | | | | | Orally |
| Gentian | | | | | Sweet Taste Changing |
| | | | | | to Intensely Bitter |
| | | | | | Stimulate Appetite |
| Ginger | g | 0.26-10 | 0.7-5 | 2.17 | Flush, rosy complexion |
| | | | | | Stimulate Appetite |
| | | | | | Aromatic Smell |
| Ginseng | g | 0.5-20 | 1.1-2.9 | 1.6 | Clean Taste |
| | | | | | Energetic feeling |
| Holy thistle | | | | | Stimulate Appetite |
| | | | | | Bitter Taste |
| Hops | mg | 1-6,000 | 178-2,990 | 934 | Calming |
| | | | | | Stimulate Appetite |
| | | | | | Aids Withdrawal |
| Horehound | | | | | Bitter Taste |
| | | | | | Calming |
| Lemon Peel, | | | | | Stimulate Appetite |
| dried (Citri | | | | | Spicy, Acidic Taste |
| pericardium) | | | | | |
| Mugwort | | | | | Stimulate Appetite |
| | | | | | Relieves Flatulence |
| | | | | | Associated with |
| | | | | | Carbonation |
| | | | | | Spicy Taste |
| Orange (unripe) | | | | | Stimulate Appetite |
| | | | | | Bitter Aromatic |
| Peppermint | | | | | Stimulate Appetite |
| | | | | | Relieves Flatulence |
| | | | | | Associated with |
| | | | | | Carbonation |
| | | | | | Calming |
| Quassia | | | | | Stimulate Appetite |
| | | | | | Intensely Bitter |
| Red sage | | | | | Stimulate Appetite |
| | | | | | Relieves Flatulence |
| | | | | | Associated with |
| | | | | | Carbonation |
| | | | | | Spicy and Bitter Taste |
| Rosemary | | | | | Stimulate Appetite |
| | | | | | Relieves Flatulence |
| | | | | | Associated with |
| | | | | | Carbonation |
| | | | | | Pungent Taste |
| Star Anise | | | | | Stimulate Appetite |
| | | | | | Pungent and Spicy |
| Thyme | | | | | Stimulate Appetite |
| | | | | | Relieves Flatulence |
| | | | | | Associated with |
| | | | | | Carbonation |
| | | | | | Pungent Taste |
| Tumeric | | | | | Stimulate Appetite |
| | | | | | Relieves Flatulence |
| | | | | | Associated with |
| | | | | | Carbonation |
| | | | | | Pungent Bitter Taste |
| Vitamin K | μg | 12-1,950 | 80-990 | 426 | Feeling of Warmth from |
| | | | | | Accumulated Mega- |
| | | | | | amounts |
| Wormwood | | | | | Stimulate Appetite |
| | | | | | Bitter Aromatic, Intense |
| | | | | | Taste |
| | | | | | Strongly Relieves |
| | | | | | Flatulence associated |
| | | | | | with Carbonation |
| Yarrow | | | | | Stimulate Appetite |
| | | | | | Relieves Flatulence |
| | | | | | Associated with |
| | | | | | Carbonation |
| Zinc and zinc | | 1-120 mg | 5-64 mg | 22 mg | Energetic feeling |
| picolinate or | | | | | |
| polynicotinate | | | | | |
| equivalents | | | | | |

More Preferred Short Term Psychological Feedback Substituents: In a more preferred embodiment, the compositions of the present invention will contain one or more preferred short term psychological feedback substituents, such more preferred substituents being selected from the group consisting of: Anandamide; Alcohol Enhancers, Standard Flavorants; Angelica root; Boldo; California Poppy; Capsaicum; Cayenne; GABA; Gentian; Tumeric; Vitamin K; and Wormwood.

Highly Preferred Short Term Psychological Feedback Substituents: In a still more preferred embodiment, the compositions of the present invention will contain one or more highly preferred short term psychological feedback substituents, such highly preferred substituents being selected from the group consisting of: an anandamide; an alcohol enhancer; Angelica root; Boldo; California Poppy; Capsaicum; GABA; Gentian; Tumeric; Vitamin K; and Wormwood.

As indicated above, the invention specifically contemplates compositions containing one or more substituents of substituent classes: (A) that provide nutritional benefit for bodybuilding, (B) that provide traditional psychological feedback, (C) that provide long term psychological feedback, and/or (D) that provide short term psychological feedback. Thus, the invention specifically contemplates compositions containing at least one substituent of each of the substituent classes: (A), (B), (C) and (D); (A), (B) and (C); (A), (B) and (D); (A), (C) and (D); (B), (C) and (D); (A) and (B); (A) and (C); (A) and (D); (B) and (C); (B) and (D); and (C) and (D).

The invention further contemplates compositions containing more than one substituent of each of the following substituent classes: (A), (B), (C) and (D); (A), (B) and (C); (A), (B) and (D); (A), (C) and (D); (B), (C) and (D); (A) and (B); (A) and (C); (A) and (D); (B) and (C); (B) and (D); (C) and (D); (A); (B); (C); and (D).

The invention thus specifically contemplates compositions containing: one or more of the above-recited preferred nutritionally beneficial substituents (A), one or more of the above-recited preferred long term psychological feedback substituents (C), and one or more of the above-recited preferred short term psychological feedback substituents (D).

The invention additionally contemplates preferred compositions containing either one or more of the above-recited more preferred nutritionally beneficial substituents (A) or one or more of the above-recited highly preferred nutritionally beneficial substituents (A), and one or more of the above-recited preferred long term psychological feedback substituents (B), and one or more of the above-recited preferred short term psychological feedback substituents (C).

The invention additionally contemplates preferred compositions containing one or more of the above-recited preferred nutritionally beneficial substituents (A), and one or more of the above-recited preferred long term psychological feedback substituents (C), and either one or more of the above-recited more preferred short term psychological feedback substituents (D) or one or more of the above-recited highly preferred short term psychological feedback substituents (D).

The invention additionally contemplates preferred compositions containing one or more of the above-recited preferred nutritionally beneficial substituents (A), and either one or more of the above-recited more preferred long term psychological feedback substituents (C) or one or more of the above-recited highly preferred long term psychological feedback substituents (C), and one or more of the above-recited preferred short term psychological feedback substituents (D).

The invention further contemplates more preferred compositions containing one or more of the above-recited more preferred nutritionally beneficial substituents (A), and one or more of the above-recited preferred long term psychological feedback substituents (C), and one or more of the above-recited more preferred short term psychological feedback substituents (D).

The invention further contemplates more preferred compositions containing one or more of the above-recited preferred nutritionally beneficial substituents (A), and one or more of the above-recited more preferred long term psychological feedback substituents (C), and one or more of the above-recited more preferred short term psychological feedback substituents (D).

The invention further contemplates highly preferred compositions containing one or more of the above-recited highly preferred nutritionally beneficial substituents (A), and one or more of the above-recited preferred long term psychological feedback substituents (C), and one or more of the above-recited highly preferred short term psychological feedback substituents (D).

The invention further contemplates highly preferred compositions containing one or more of the above-recited highly preferred nutritionally beneficial substituents (A), and one or more of the above-recited highly preferred long term psychological feedback substituents (C), and one or more of the above-recited preferred short term psychological feedback substituents (D).

The invention further contemplates highly preferred compositions containing one or more of the above-recited preferred nutritionally beneficial substituents (A), and one or more of the above-recited highly preferred long term psychological feedback substituents (C), and one or more of the above-recited highly preferred short term psychological feedback substituents (D).

The invention further contemplates very highly preferred compositions containing one or more of the above-recited highly preferred nutritionally beneficial substituents (A), and one or more of the above-recited highly preferred long term psychological feedback substituents (C), and one or more of the above-recited highly preferred short term psychological feedback substituents (D).

In all such compositions, traditional psychological feedback substituents (B) may be present.

In accordance with the invention, the compositions of the present invention may additionally contain non-pharmacological agents such as stomach acid buffering compounds, antacids, fiber, laxatives, muscle relaxants, analgesics, acetaminophens, ibuprophens, topical antibiotics, anti-inflammatory agents, etc.) at non-prescription levels. In yet another embodiment, the compositions of the invention may be formulated to contain prescription pharmacological agents, such as hormones (e.g., insulin, thyroid hormone, etc.), anti-inflammatory agents, hypertensives, anti-arrhythmias, etc. The co-administration of the compositions of the present invention and such prescription pharmacological agents has the salutary effect of confirming or the consumer's perception or recollection that a dosage has been consumed or administered, and as such aids in preventing overdosing and underdosing by individuals who are self administering such pharmacological agents.

As stated above, in a particularly preferred embodiment, the compositions of the present invention are provided via vehicles for oral or translingual delivery.

In a preferred embodiment, such vehicles are formulated to contain one or more additional substituents selected from the group consisting of: a xanthine alkaloid (such as a methylxanthine (caffeine, theophylline, theobromine, etc.) obtainable synthetically, or by extraction of plants (e.g. coffee beans, cola nuts, tea plants, etc.); carbonic acid, milk, vegetable juice, fruit juice, phosphoric acid, citric acid, hops, cocoa, chocolate, anandamide, quinine and malic acid.

The amounts of methylxanthines (and especially caffeine) present in the compositions of the invention are designed to result in the delivery of an amount of such methylxanthines sufficient to impart the desired effect. Thus, where the composition contain compounds that interact (or sequester) such methylxanthines, proportionately larger amounts of methylxanthine will be included in the composition. Likewise, where the composition contain substituents that enhance the desired effect of a methylxanthine, the amount of the methyixanthine in the composition will be proportionately reduced.

Serotonin, and its precursors, and substituents that stimulate serotonin production and/or release are also preferred substituents of the compositions of the present invention.

The orally or translingually delivered compositions of the present invention may comprise clear solutions, or may be suspensions of particulate material (such as plant pulp or other tissue). When provided as a beverage, such beverages are typically free-flowing and have a viscosity similar to that of water. Preferably, thickening agents are added to impart greater viscosity to the beverages. The beverages of the present invention are typically dispensed and stored in containers (e.g., cardboard containers, glass or plastic bottles, metal cans, etc.).

### USES OF THE COMPOSITIONS OF THE INVENTION

The compositions of the present invention provide a nutritionally beneficial substituent to an individual in a manner accompanied by a reinforcing psychological feedback sensation. The psychological feedback sensation imparts an immediate physiological recognition of the composition (such as a feeling of warmth, or a flush feeling) as well as a long term psychological feedback (such as a counter-depressive effect or a sense of well-being). These feedbacks become psychologically associated with the nutritional benefit of the composition and increase an individual's desire to take or use the composition on a regular basis, and thus serve to ensure that the individual obtains a nutritionally beneficial amount of the composition.

The compositions of the present invention are formulated to provide nutritionally beneficial amounts of substituents that facilitate or enhance bodybuilding. In addition to the classes of substituents identified above, the compositions of the invention can be formulated to contain vitamins, minerals, co-factors, etc. in amounts sufficient to improve the general health of the recipient.

While various embodiments of the present invention have been described above, it should be understood that they have been presented by way of example only, and not limitation. Thus, the breadth and scope of the present invention should not be limited by any of the above-described exemplary embodiments, but should be defined only in accordance with the following claims and their equivalents.

What is claimed is:

1. A composition for human consumption, comprising:
a nutritionally beneficial substituent (A) selected from the group consisting of:
creatine monohydrate; HMB; L-glutamine; taurine; whey peptides; chromium; potassium; phosphorus; and magnesium;
said substituent present in an amount sufficient to enhance or facilitate bodybuilding; and at least one additional substituent (B) that provides traditional psychological feedback selected from the group consisting of
caffeine or a caffeine equivalent; tryptophan; ephedra; cola; green tea extract; carbonic acid; phosphoric acid; citric acid; hops; cocoa; chocolate; an anandamide; quinine; malic acid; a sweetener; a fruit juice or fruit juice extract; milk; a vegetable juice or vegetable juice extract; kudzu and 5-hydroxy-tryptophan;
present in an amount sufficient to provide a sensory psychological feedback.

2. The composition of claim 1, wherein said composition is a beverage.

3. The composition of claim 2, wherein said beverage is substantially alcohol-free.

4. The compositions of any of claims 1-3 which additionally contains at least one additional substituent (C) that provides long term psychological feedback substituent or at least one additional substituent (D) that provides short term psychological feedback; wherein
said substituent (C) that provides said long term psychological feedback is selected from the group consisting of:
an anandamide; 5-hydroxytryptophan; 5-fluoro-A-methyltryptamine; 5-fluorotryptophan; 6-fluorotryptophan; tryptophan; allocryptine; caffeine; theophylline; theobromine; California poppy; calcium; chromium picolinate; chromium polynicotinate; chicalote extract; cocoa; chocolate; Damiana (Turnera diffusa); DL-phenylalanine; ephedra; ephedrine; epinephrine; GABA; ginger; ginseng; L-glutamine; green tea; guarana; kava kava; lactuca virosa; L-tyrosine; lobelia; magnesium; maraba; protopine; pseudophedrine; pseudoepinephrine; pyridoxal-5-phosphate; red rice yeast; serotonin; sucrose; fructose; glucose; high fructose corn syrup; and St. Johnswort; and
is present in an amount sufficient to provide a long term feeling of well-being or calmness; and
said substituent (D) that provides said short term psychological feedback is selected from the group consisting of:
an anandamide; an alcohol enhancer; angelica root; balm; bitter orange (Auranti pericarpium); bogbean; boldo; calamus; California poppy; capsaicum; caraway; cayenne; chamomile; cinchona bark; quinine; chocolate; cinnamon; clove; cocoa; condurango; dandelion; elecampane; GABA; gentian; ginger; ginseng; holy thistle; hops; horehound; dried lemon peel (Citri pericardium); mugwort; unripe orange; peppermint; quassia; red sage; rosemary; star anise; thyme; tumeric; wormwood; yarrow; and zinc; and
is present in an amount sufficient to provide a short term sensation of warmth, tingling, excitement, tranquility and well-being, or a distinctive, intense, bitter or unusual taste.

5. The composition of claim 4, wherein said composition contains at least one of said substituent (C) that provides said long term psychological feedback.

6. The composition of claim 4, wherein said composition contains at least one of said substituent (D) that provides said short term psychological feedback.

7. The composition of claim 4, wherein said composition contains both at least one of said substituent (C) that provides said long term psychological feedback, and at least one of said substituent (D) that provides said short term psychological feedback.

8. The composition of any of claims 1-3, wherein said composition contains at least two of said nutritionally beneficial substituents (A).

9. The composition of claim 4, wherein said composition contains at least two of said nutritionally beneficial substituents (A).

10. The composition of claim 9, wherein said composition contains at least four of said nutritionally beneficial substituents (A).

11. The composition of claim 5, wherein said composition contains at least two of said nutritionally beneficial substituents (A).

12. The composition of claim 11, wherein said composition contains at least four of said nutritionally beneficial substituents (A).

13. The composition of claim 6, wherein said composition contains at least two of said nutritionally beneficial substituents (A)

14. The composition of claim 13, wherein said composition contains at least four of said nutritionally beneficial substituents (A).

15. The composition of claim 7, wherein said composition contains at least two of said nutritionally beneficial substituents (A).

16. The composition of claim 15, wherein said composition contains at least four of said nutritionally beneficial substituents (A).

17. The beverage composition of claim 2, wherein said nutritionally beneficial substituent is creatine monohydrate.

18. The beverage composition of claim 2, wherein said nutritionally beneficial substituent is HMB.

19. The beverage composition of claim 2, wherein said nutritionally beneficial substituent is L-glutamine.

20. The beverage composition of claim 2, wherein said nutritionally beneficial substituent is taurine.

21. The beverage composition of claim 2, wherein said nutritionally beneficial substituents are selected from the group consisting of creatine monohydrate, HMB, L-glutamine, and taurine.

22. The composition of claim 4, wherein said substituent (C) is selected from the group consisting of 5-hydroxy-tryptophan, 5-fluoro-A-methyltrypamine, 5-fluoro-tryptophan, L-glutamic acid, L-phenylaianine, and L-tryptophan.

23. The composition of claim 4, wherein said substituent (C) is selected from the group consisting of enkephaline, ephinephrine, and serotonin.

24. The composition of claim 4, wherein said substituent (C) is selected from the group consisting of red rice yeast, damiana, ephedra, ginger, ginseng, goto kola, lobelia, ma huang, maraba and psilocybin.

25. The composition of claim 4, wherein said composition contains more than one of said long term psychological feedback substituents (C).

26. The composition of claim 5, wherein said composition contains more than one of said long term psychological feedback substituents (C).

27. The composition of claim 6, wherein said composition contains more than one of said long term psychological feedback substituents (C).

28. The composition of claim 7, wherein said composition contains more than one of said long term psychological feedback substituents (C).

29. The composition of claim 4, wherein said composition contains a substituent (D) selected from the group consisting of capsaicum and cayenne.

30. The composition of claim 4, wherein said composition contains a substituent (D) selected from the group consisting of ginger and ginseng.

31. The composition of claim 4, wherein said composition contains more than one of said short term psychological feedback substituents (D).

32. The composition of claim 5, wherein said composition contains more than one of said long term psychological feedback substituents (D).

33. The composition of claim 6, wherein said composition contains more than one of said long term psychological feedback substituents (D).

34. The composition of claim 7, wherein said composition contains more than one of said long term psychological feedback substituents (D).

35. The composition of claim 4, wherein said composition is a beverage, and wherein said substituent (B) of said beverage is caffeine.

36. The composition of claim 4, wherein said composition is a beverage, and wherein said substituent (B) of said beverage is cola.

37. The composition of claim 4, wherein said composition is a beverage, and wherein said substituent (B) of said beverage is carbonic acid.

38. The composition of claim 4, wherein said composition is a beverage, and wherein said substituent (B) of said beverage is phosphoric acid.

39. The composition of claim 4, wherein said composition is a beverage, and wherein said substituent (B) of said beverage is citric acid.

40. The composition of claim 4, wherein said composition is a beverage, and wherein said substituent (B) of said beverage is hops.

41. The composition of claim 4, wherein said composition is a beverage, and wherein said substituent (B) of said beverage is selected from the group consisting of cocoa, chocolate and an anandamide.

42. The composition of claim 4, wherein said composition is a beverage, and wherein said substituent (B) of said beverage is malic acid.

43. The composition of claim 4, wherein said composition is a beverage, and wherein said substituent (B) of said beverage is quinine.

44. The composition of claim 4, wherein said composition is a beverage, and wherein said substituent (B) of said beverage is a sweetener.

45. The beverage composition of claim 44, wherein said sweetener is selected from the group consisting of fructose, high fructose corn syrup, sucrose, maltose, glucose, lactose, sorbitol, and galactose.

46. The beverage composition of claim 44, wherein said sweetener is selected from the group consisting of aspartame, saccharin, an L-sugar, and a cyclamate.

47. The composition of claim 4, wherein said composition is a beverage, and wherein said substituent (B) of said beverage is a fruit juice or fruit juice extract.

48. The beverage composition of claim 47, wherein said fruit juice or fruit juice extract comprises a juice or extract from a fruit selected from the group consisting of apple, pineapple, grape, pear, banana, plum, cherry, peach, strawberry, blueberry, cranberry, blackberry, orange, grapefruit, lemon, and lime.

49. The composition of claim 4, wherein said composition is a beverage, and wherein said substituent (B) of said beverage is milk.

50. The composition of claim 4, wherein said composition is a beverage, and wherein said substituent (B) of said beverage is a vegetable juice or vegetable juice extract.

51. A composition for human consumption, comprising:
at least one nutritionally beneficial substituent (A) selected from the group consisting of:
creatine monohydrate; HMB; L-glutamine; taurine; whey peptides; chromium; potassium; phosphorus; and magnesium;
said substituent being present in an amount sufficient to enhance or facilitate bodybuilding of said human recipient; and
at least one additional substituent (C) that provides long term psychological feedback substituent or at least one additional substituent (D) that provides short term psychological feedback;
wherein said substituent (C) that provides said long term psychological feedback is selected from the group consisting of:
an anandamide; 5-hydroxytryptophan; 5-fluoro-A-methyltryptamine; 5-fluorotryptophan; 6-fluorotryptophan;
tryptophan; allocryptine; caffeine; theophylline; theobromine; California poppy; calcium; chromium picolinate; chromium polynicotinate; chicalote extract; cocoa; chocolate; Damiana (Turnera diffusa); DL-phenylalanine; ephedra; ephedrine; epinephrine; GABA; ginger; ginseng; L-glutamine; green tea; guarana; kava kava; lactuca virosa; L-tyrosine; lobelia; magnesium; maraba; protopine; pseudophedrine; pseudoepinephrine; pyridoxal-5-phosphate; red rice yeast; serotonin; sucrose; fructose; glucose; high fructose corn syrup; and St. Johnswort; and
is present in an amount sufficient to provide a long term feeling of well-being or calmness; and
said substituent (D) that provides said short term psychological feedback is selected from the group consisting of:
an anandamide; an alcohol enhancer; angelica root; balm; bitter orange (Auranti pericarpium); bogbean; boldo; calamus; California poppy; capsaicum; caraway; cayenne; chamomile; cinchona bark; quinine; chocolate; cinnamon; clove; cocoa; condurango; dandelion; elecampane; GABA; gentian; ginger; ginseng; holy thistle; hops; horehound; dried lemon peel (Citri perlcardium); mugwort; unripe orange; peppermint; quassia; red sage; rosemary; star anise; thyme; tumeric; wormwood; yarrow; and zinc; and
is present in an amount sufficient to provide a short term sensation of warmth, tingling, excitement, tranquility and well-being, or a distinctive, intense, bitter or unusual taste.

52. The composition of claim 51, wherein said composition contains at least one of said substituent (C) that provides said long term psychological feedback.

53. The composition of claim 51, wherein said composition contains at least one of said substituent (D) that provides said short term psychological feedback.

54. The composition of claim 51, wherein said composition contains both at least one of said substituent (C) that provides said long term psychological feedback, and at least one of said substituent (D) that provides said short term psychological feedback.

55. A method for enhancing or facilitating bodybuilding in a human, which comprises administering or providing to said human a composition comprising:
at least one nutritionally beneficial substituent (A) selected from the group consisting of:
creatine monohydrate; HMB; L-glutamine; taurine; whey peptides; chromium; potassium; phosphorus; and magnesium;
said substituent being present in an amount sufficient to enhance or facilitate bodybuilding of said human recipient; and
at least one additional substituent (C) that provides long term psychological feedback substituent or at least one additional substituent (D) that provides short term psychological feedback;
wherein said substituent (C) that provides said long term psychological feedback is selected from the group consisting of:
an anandamide; 5-hydroxytryptophan; 5-fluoro-A-methyltryptamine; 5-fluorotryptophan; 6-fluorotryptophan; tryptophan; allocryptine; caffeine; theophylline; theobromine; California poppy; calcium; chromium picolinate; chromium polynicotinate; chicalote extract; cocoa; chocolate; Damiana (Turnera diffusa); DL-phenylalanine; ephedra; ephedrine; epinephrine; GABA; ginger; ginseng; L-glutamine; green tea; guarana; kava kava; lactuca virosa; L-tyrosine; lobelia; magnesium; maraba; protopine; pseudophedrine; pseudoepinephrine; pyridoxal-5-phosphate; red rice yeast; serotonin; sucrose; fructose; glucose; high fructose corn syrup; and St. Johnswort; and
is present in an amount sufficient to provide a long term feeling of well-being or calmness; and
said substituent (D) that provides said short term psychological feedback is selected from the group consisting of:
an anandamide; an alcohol enhancer; angelica root; balm; bitter orange (Auranti pericarpium); bogbean; boldo; calamus; California poppy; capsaicum; caraway; cayenne; chamomile; cinchona bark; quinine; chocolate; cinnamon; clove; cocoa; condurango; dandelion; elecampane; GABA; gentian; ginger; ginseng; holy thistle; hops; horehound; dried lemon peel (Citri pericardium); mugwort; unripe orange; peppermint; quassia; red sage; rosemary; star anise; thyme; tumeric; wormwood; yarrow; and zinc; and
is present in an amount sufficient to provide a short term sensation of warmth, tingling, excitement, tranquility and well-being, or a distinctive, intense, bitter or unusual taste.

56. The method of claim 55, wherein said composition contains at least one of said substituent (C) that provides said long term psychological feedback.

57. The method of claim 55, wherein said composition contains at least one of said substituent (D) that provides said short term psychological feedback.

58. The method of claim 55, wherein said composition contains both at least one of said substituent (C) that provides said long term psychological feedback, and at least one of said substituent (D) that provides said short term psychological feedback.

59. The method of claim 56, wherein said composition contains two of said substituent (C) that provides said long term psychological feedback.

60. The method of claim 56, wherein said composition contains three of said substituent (C) that provides said long term psychological feedback.

61. The method of claim 56, wherein said composition contains four of said substituent (C) that provides said long term psychological feedback.

62. The method of claim 56, wherein said composition contains five of said substituent (C) that provides said long term psychological feedback.
